(19) United States
(12) Patent Application Publication
(10) Pub. No.: US 2026/0041407 A1
(43) Pub. Date: Feb. 12, 2026

(54) SYSTEM AND METHOD FOR INTELLIGENT ULTRASOUND DATA ACQUISITION WITH WEARABLE ULTRASOUND PROBE HAVING DISTRIBUTED ELECTRONICS

(71) Applicant: GE Precision Healthcare LLC, Waukesha, WI (US)

(72) Inventors: Warren Lee, Niskayuna, NY (US); David Shoudy, Niskayuna, NY (US); Aaron Dentinger, Latham, NY (US); Kirk Wallace, Glenville, NY (US); David M. Mills, Niskayuna, NY (US)

(21) Appl. No.: 18/800,439

(22) Filed: Aug. 12, 2024

(51) Int. Cl. A61B 8/00

(52) U.S. Cl. CPC A61B 8/543; A61B 8/4227; A61B 8/4236

(57) ABSTRACT

Systems and methods for performing intelligent ultrasound data acquisition with a wearable ultrasound probe having electronics distributed between a patient-worn probe transducer assembly and a patient-worn probe controller assembly are provided. The wearable ultrasound probe includes a patient-worn probe transducer assembly including transducer elements disposed in a first housing. The transducer elements are configured to transmit ultrasound beams and convert received echoes to electrical signals in response to control signals. The wearable ultrasound probe includes a patient-worn probe controller assembly including at least one control processor disposed in a second housing. The at least one control processor is communicatively coupled to a connected device. The at least one control processor is communicatively coupled to the patient-worn probe transducer assembly via a wired connection. The at least one control processor is configured to provide the control signals to the patient-worn probe transducer assembly for controlling ultrasound data acquisition.

### FIELD

Certain embodiments relate to ultrasound imaging. More specifically, certain embodiments relate to a method and system for performing intelligent ultrasound data acquisition with a wearable ultrasound probe having electronics distributed between a patient-worn probe transducer assembly and a patient-worn probe controller assembly. As referred to herein, intelligent ultrasound data acquisition refers to reducing power consumption by using non-ultrasound sensor data to selectively perform ultrasound acquisition at specific times and locations.

### BACKGROUND

Ultrasound imaging is a medical imaging technique for imaging organs and soft tissues in a human body. Ultrasound imaging uses non-invasive high frequency sound waves to produce one-dimensional (1D), two-dimensional (2D), three-dimensional (3D), and/or four-dimensional (4D) (i.e., real-time/continuous 3D images) images in real time.

Ultrasound examinations are typically performed by an ultrasound operator placing an ultrasound transducer on a body surface of a patient and manipulating the ultrasound transducer about the body surface to manually direct the acquisition of ultrasound image data. The manual manipulation of the ultrasound transducer is not ideal for prolonged ultrasound image data acquisition. Instead, an ultrasound probe, such as a patch probe or the like, may be secured in a fixed position on the body surface of a patient for ultrasound image data acquisition over an extended period of time. However, the footprint of the ultrasound probe may be undesirably large because both the transducer and associated electronics are disposed within the housing of the probe. The large footprint may render the probe unsuitable for long-term wear because it interferes with normal patient activities. Furthermore, the large footprint of the ultrasound probe may inhibit optimal placement of probe when the acoustic window is small, such as between ribs of a patient for cardiac imaging. In addition, the ultrasound probe may easily be dislodged if the probe cable is accidentally pulled, thereby moving the probe out of alignment with the desired anatomy. Wearable ultrasound probes also suffer from power consumption and heating issues due to the continuous acquisition of ultrasound data.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for performing intelligent ultrasound data acquisition with a wearable ultrasound probe having electronics distributed between a patient-worn probe transducer assembly and a patient-worn probe controller assembly, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system comprising a wearable ultrasound probe having electronics distributed between a patient-worn probe transducer assembly and a patient-worn probe controller assembly, the wearable ultrasound probe operable to perform intelligent ultrasound data acquisition and communicatively coupled by a wired connection to a connected device, in accordance with various embodiments.

FIG. 2 is a block diagram of an exemplary ultrasound system comprising a wearable ultrasound probe having electronics distributed between a patient-worn probe transducer assembly and a patient-worn probe controller assembly, the wearable ultrasound probe operable to perform intelligent ultrasound data acquisition and communicatively coupled by a wireless connection to a connected device, in accordance with various embodiments.

FIG. 3 is an illustration of an exemplary wearable ultrasound probe having electronics distributed between a patient-worn probe transducer assembly and a patient-worn probe controller assembly, the wearable ultrasound probe affixed to a patient, in accordance with various embodiments.

FIG. 4 is an exemplary display of an electrocardiogram (ECG) signal acquired by a non-ultrasound sensor and a comparison of the timing of continuously acquired ultrasound data versus exemplary ECG triggered ultrasound data acquisitions, in accordance with various embodiments.

FIG. 5 is an exemplary display of a respiration sensor signal acquired by a non-ultrasound sensor and exemplary partial ultrasound volume acquisitions determined by a location of the target anatomical structure at different respiratory phases, in accordance with various embodiments.

FIG. 6 is an exemplary display of a local pulse oximeter sensor signal acquired by a non-ultrasound sensor and indicative of local blood flow near a skin surface analyzed to determine gating of ultrasound acquisitions, in accordance with various embodiments.

FIG. 7 is a flow chart illustrating exemplary steps that may be utilized for performing intelligent ultrasound data acquisition with a wearable ultrasound probe having electronics distributed between a patient-worn probe transducer assembly and a patient-worn probe controller assembly, in accordance with various embodiments.

### DETAILED DESCRIPTION

Certain embodiments may be found in a method and system for performing intelligent ultrasound data acquisition with a wearable ultrasound probe having electronics distributed between a patient-worn probe transducer assembly and a patient-worn probe controller assembly. Aspects of the present disclosure have the technical effect of reducing a size of a patient-worn probe transducer assembly by splitting electronics between multiple patient-worn housings. Various embodiments have the technical effect of simplifying placement of a patient-worn probe transducer assembly when the acoustic window is small, such as between ribs of a patient for cardiac imaging, by reducing the size of the patient-worn probe transducer assembly. Certain embodiments have the technical effect of preventing dislodgement of a patient-worn probe transducer assembly by having a primary fastening of the patient-worn probe transducer assembly and a separate, secondary fastening a short distance away of a patient-worn probe controller assembly, such that any accidental pulling of a cable connecting the wearable ultrasound probe to a connected device would result in the secondary fastening being pulled first while the patient-worn probe transducer assembly having the transducer elements remains properly fixed to the patient. Aspects of the present disclosure have the technical effect of reducing power consumption by using non-ultrasound sensor data to selectively perform ultrasound acquisition at specific times and locations (i.e., intelligent ultrasound acquisition). The reduced power consumption increases an amount of time scanning can be performed due to improved battery life and reduces ultrasound probe heating issues. Various embodiments have the technical effect of simplifying patient care by consolidating multiple physiological monitors into a single wearable ultrasound probe. Certain embodiments have the technical effect of improving patient care by implementing physiological sensors to enable more accurate automated detection, tracking, and analysis of anatomies to estimate desired clinical parameters.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random access memory, hard disk, or the like) or multiple pieces of hardware. Similarly, the programs may be standalone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized, and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising”, “including”, or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode, which can be one-dimensional (1D), two-dimensional (2D), three-dimensional (3D), or four-dimensional (4D), and comprising Brightness mode (B-mode), Motion mode (M-mode), Color Motion mode (CM-mode), Color Flow mode (CF-mode), Pulsed Wave (PW) Doppler, Continuous Wave (CW) Doppler, Contrast Enhanced Ultrasound (CEUS), and/or sub-modes of B-mode and/or CF-mode such as Harmonic Imaging, Shear Wave Elasticity Imaging (SWEI), Strain Elastography, Tissue Velocity Imaging (TVI), Power Doppler Imaging (PDI), B-flow, Micro Vascular Imaging (MVI), Ultrasound-Guided Attenuation Parameter (UGAP), and the like.

Furthermore, the term processor or processing unit, as used herein, refers to any type of processing unit that can carry out the required calculations needed for the various embodiments, such as single or multi-core Central Processing Unit (CPU), Accelerated Processing Unit (APU), Graphic Processing Unit (GPU), Digital Signal Processor (DSP), Field Programmable Gate Array (FPGA), System on a Chip (SoC), Application-Specific Integrated Circuit (ASIC), or a combination thereof.

In various embodiments, ultrasound processing to form images is performed, for example, including ultrasound beamforming, such as receive beamforming, in software, firmware, hardware, or a combination thereof.

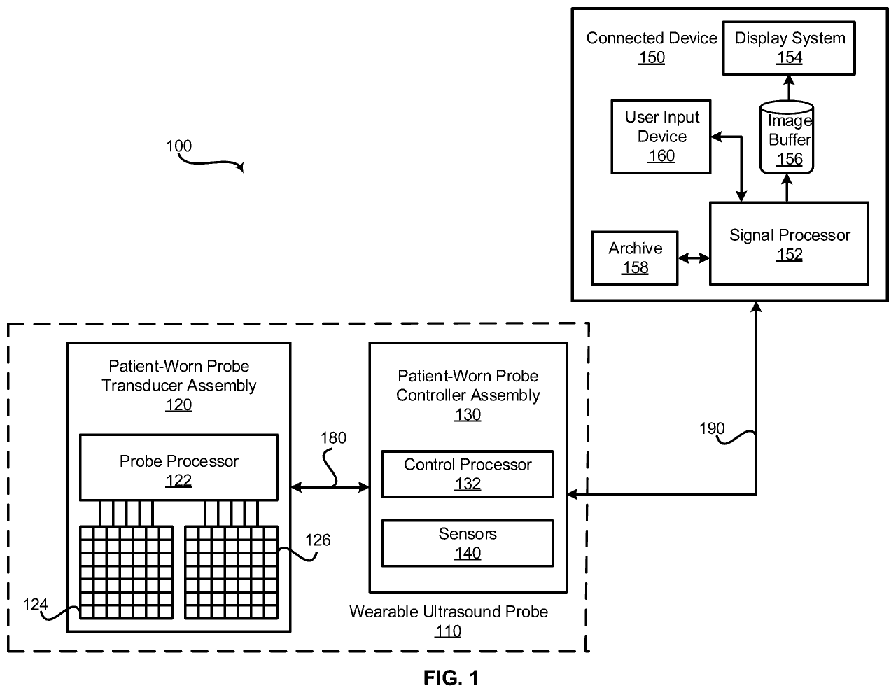

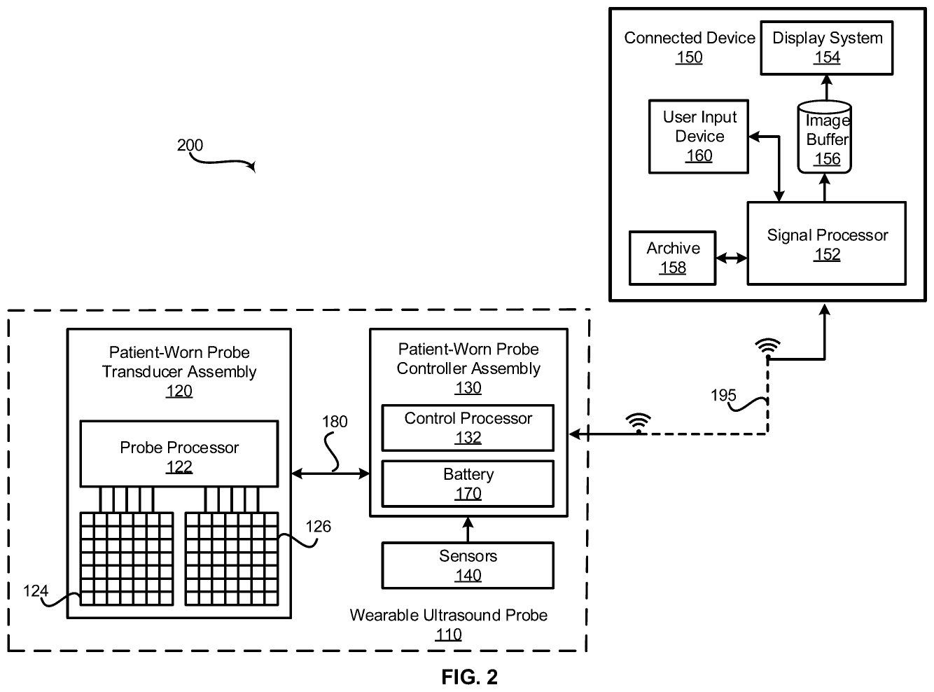

FIG. 1 is a block diagram of an exemplary ultrasound system 100 comprising a wearable ultrasound probe 110 having electronics 122, 124, 126, 132, 140 distributed between a patient-worn probe transducer assembly 120 and a patient-worn probe controller assembly 130, the wearable ultrasound probe 110 operable to perform intelligent ultrasound data acquisition and communicatively coupled by a wired connection 190 to a connected device 150, in accordance with various embodiments. FIG. 2 is a block diagram of an exemplary ultrasound system 200 comprising a wearable ultrasound probe 110 having electronics 122, 124, 126, 132 distributed between a patient-worn probe transducer assembly 120 and a patient-worn probe controller assembly 130, the wearable ultrasound probe 110 operable to perform intelligent ultrasound data acquisition and communicatively coupled by a wireless connection 195 to a connected device 150, in accordance with various embodiments. Referring to FIGS. 1 and 2, there is shown an ultrasound system 100, 200. The ultrasound system 100, 200 comprises a wearable ultrasound probe 110 and a connected device 150. The wearable ultrasound probe 110 comprises a patient-worn probe transducer assembly 120 and a patient-worn probe controller assembly 130. The patient-worn probe transducer assembly 120 comprises transmit transducer elements 124 and receive transducer elements 126 that in various embodiments may constitute the same transducer elements. In various embodiments, the patient-worn probe transducer assembly 120 further comprises a probe processor 122. The patient-worn probe controller assembly 130 may comprise a control processor 132. The patient-worn probe transducer assembly 120 may be communicatively coupled to the patient-worn probe controller assembly 130 by a probe cable 180. In an exemplary embodiment, the wearable ultrasound probe 110 further comprises non-ultrasound sensors 140 that may be disposed in a housing of the patient-worn probe controller assembly 130 (as shown in FIG. 1), in a housing separate from the patient-worn probe transducer assembly 120 and the patient-worn probe controller assembly 130 (as shown in FIG. 2), and/or in the patient-worn probe transducer assembly 120 (not shown). In certain embodiments, such as where the wearable ultrasound probe 110 is wirelessly connected 195 to the connected device 150 (as shown in FIG. 2, for example), the wearable ultrasound probe 110 may further comprise one or more batteries 170 disposed in housing(s) of the patient-worn probe transducer assembly 120, patient-worn probe controller assembly 130, and/or in a separate housing with sensors 140. The connected device 150 may comprise a signal processor 152, a display system 154, an image buffer 156, an archive 158, and a user input device 160. The connected device 150 may be communicatively coupled to the patient-worn probe controller assembly 130 of the wearable ultrasound probe 110 by a wired connection 190 (as shown in FIG. 1) or a wireless connection 195 (as shown in FIG. 2).

The wearable ultrasound probe 110 may be a phased array, linear array, curved array, or any suitable shape or combination of shapes. The wearable ultrasound probe 110 may be operable to acquire one-dimensional (1D), two-dimensional (2D), three-dimensional (3D) probe, or four-dimensional (4D) ultrasound data. In an exemplary embodiment, the wearable ultrasound probe 110 may be an electronic 4D (e4D) probe or any suitable wearable ultrasound probe. The wearable ultrasound probe 110 may comprise a patient-worn probe transducer assembly 120 and a patient-worn probe controller assembly 130. Electronics, such as transducer elements 124, 126 and processors 122, 132, may be distributed in the housings of the patient-worn probe transducer assembly 120 and the patient-worn probe controller assembly 130 to reduce a size of the housing having the transducer elements 124, 126 relative to the size of known wearable ultrasound probes. In a representative embodiment, the patient-worn probe transducer assembly 120 at least comprises transducer elements 124, 126 disposed in a housing. In various embodiments, such as when the wearable ultrasound probe 110 is an electronic 4D (e4D) probe, the patient-worn probe transducer assembly 120 further comprises a probe processor 122. In various embodiments, the patient-worn probe transducer assembly 120 may optionally include sensors 140 and/or a battery 170. However, in a preferred embodiment, the components disposed in the housing of the patient-worn probe transducer assembly 120 may be minimized to lessen a footprint of the patient-worn probe transducer assembly 120. The small footprint of the patient-worn probe transducer assembly 120 allows for simplified placement of the patient-worn probe transducer assembly 120 when the acoustic window is small, such as between ribs of a patient for cardiac imaging. The small footprint of the patient-worn probe transducer assembly 120 also allows for a reduced weight low-profile device with better user tolerability for body-worn ultrasound. The reduced weight and low-profile increase stability on the body and reduce likelihood of catching or bumping the patient-worn probe transducer assembly 120 during normal activities. The patient-worn probe controller assembly 130 at least comprises a control processor 132 disposed in a housing of the patient-worn probe controller assembly 130. In an exemplary embodiment, the patient worn probe controller assembly 130 may optionally include sensors 140 and/or a battery 170 disposed in the housing. The wearable ultrasound probe 110 may further comprise a separate patient-worn housing comprising sensors 140 and/or a battery 170.

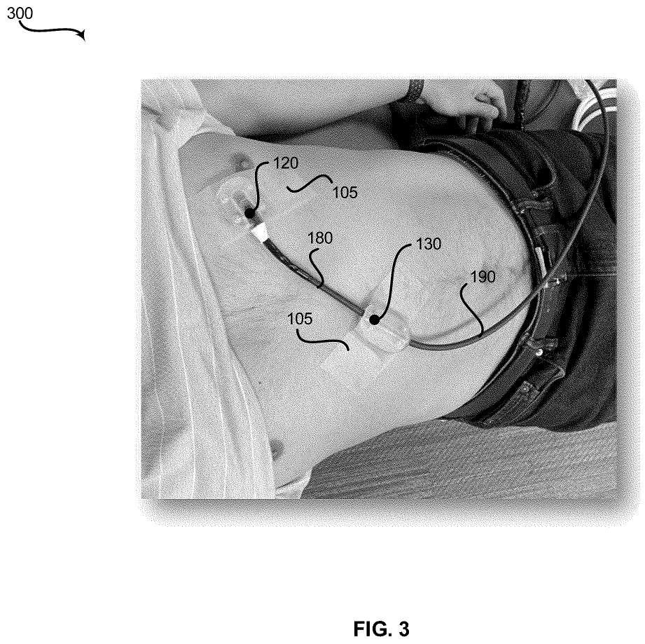

The wearable ultrasound probe 110 is configured to be worn by a patient. For example, each of the patient-worn probe transducer assembly 120 and the patient-worn probe controller assembly 130 is configured to be attached to a patient via a patient attachment mechanism, such as a fixation adhesive, a strap, a harness, and/or any suitable patient attachment mechanism. FIG. 3 is an illustration of an exemplary wearable ultrasound probe 300 having electronics distributed between a patient-worn probe transducer assembly 120 and a patient-worn probe controller assembly 130, the wearable ultrasound probe 300 affixed 105 to a patient, in accordance with various embodiments. Referring to FIG. 3, the wearable ultrasound probe 300 comprises a patient-worn probe transducer assembly 120 communicatively coupled to a patient-worn probe controller assembly 130 by a probe cable 180. The patient-worn probe controller assembly 130 of FIG. 3 is communicatively coupled to a connected device (not shown) by a wired connection 190. Each of the patient-worn probe transducer assembly 120 and the patient-worn probe controller assembly 130 are fastened to the patient by a patient attachment mechanism 105. The arrangement of a primary fastening 105 of the patient-worn probe transducer assembly 120 and a separate, secondary fastening 105 a short distance away of a patient-worn probe controller assembly 130 helps prevent dislodgement of the patient-worn probe transducer assembly 120 due to any accidental pulling of the cable 190 connecting the wearable ultrasound probe to a connected device, which would result in the secondary fastening 105 being pulled first while the patient-worn probe transducer assembly 120 having the transducer elements remains properly fixed to the patient.

Referring again to FIGS. 1 and 2, the patient-worn probe transducer assembly 120 may comprise an array of transducer elements, such as piezoelectric elements, micromachined elements, piezoelectric micromachined ultrasound transducers (PMUT) elements, capacitive micromachined ultrasound transducers (CMUT) elements, and/or any suitable transducer elements capable of converting control signals to acoustic energy and converting acoustic energy to ultrasound signals. The patient-worn probe transducer assembly 120 may comprise a group of transmit transducer elements 124 and a group of receive transducer elements 126, that normally constitute the same elements. The group of transmit transducer elements 124 may emit ultrasonic signals into a target. In a representative embodiment, the patient-worn probe transducer assembly 120 may be operable to acquire ultrasound image data covering at least a substantial portion of an anatomy, such as a heart, fetus, blood vessels, pelvic region, or any suitable anatomical region. The transmitted ultrasonic signals may be backscattered from structures in the object of interest, like blood cells or tissue, to produce echoes. The echoes are received by the receive transducer elements 126.

The control processor 132 of the patient-worn probe controller assembly 130 may comprise suitable logic, circuitry, interfaces and/or code that may be operable to control the ultrasound acquisition performed by the patient-worn probe transducer assembly 120. The control processor 132 may be one or more Application-Specific Integrated Circuits (ASICs), one or more Field Programmable Gate Arrays (FPGA), one or more System on a Chip (SoC), and/or any suitable processing element.

The control processor 132 may comprise suitable logic, circuitry, interfaces and/or code that may be operable to communicate control signals for driving transducer elements 124, 126 of the patient-worn probe transducer assembly 120. The control processor 132 may be configured to retrieve from memory, or receive from the connected device 150, transmit and receive settings for driving the transducer elements 124, 126 of the patient-worn probe transducer assembly 120. For example, the control processor 132 may retrieve or receive transmit and receive settings such as a transmit frequency, receive frequency, waveform shape, bandwidth, aperture, waveform, focus, and/or any suitable transmit and receive settings.

In a representative embodiment, the generation of the control signals is based in part on analysis of non-ultrasound sensor data. For example, the wearable ultrasound probe 110 may comprise sensors 140, such as electrocardiogram (ECG) sensor(s) 140, respiration sensor(s) 140, pulse oximeter sensor(s) 140, motion sensor(s) 140, temperature sensor(s) 140, blood pressure sensor(s) 140, and/or any suitable non-ultrasound sensor. The sensor data acquired by sensors 140 may be analyzed at a processor of the sensor 140 and/or may be communicated to another processor for analysis, such as the control processor 132 of the patient-worn probe controller assembly 130, a signal processor 152 of a connected device 150, and/or any suitable local or remote processor. For example, a motion sensor 140 may be operable to acquire patient posture sensor signals. The patient posture sensor signals may be analyzed by a processor 132, 152 to flag unreliable ultrasound acquisitions and/or to provide the motion information to a connected device 150 for storage at archive 158 and/or display at display system 154. As another example, a temperature sensor 140 may be operable to acquire temperature sensor signals. The temperature sensor signals may be analyzed by a processor 132, 152 to provide temperature information to the connected device 150 for storage at archive 158 and/or display at display system 154. As another example, a blood pressure sensor 140 may be operable to acquire blood pressure sensor signals. The blood pressure sensor signals may be analyzed by a processor 132, 152 to provide blood pressure information to the connected device 150 for storage at archive 158 and/or display at display system 154.

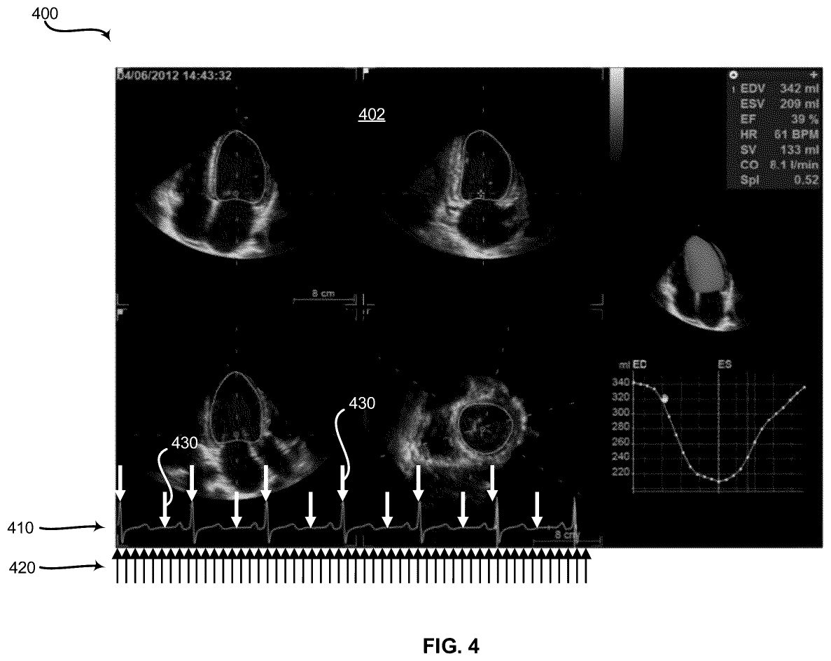

As another example, an electrocardiogram (ECG) sensor 140 may be operable to acquire ECG signals. The ECG signals may be analyzed by a processor 132, 152 to generate control signals for triggering a timing of ultrasound data acquisition. FIG. 4 is an exemplary display 400 of an electrocardiogram (ECG) signal 410 acquired by a non-ultrasound sensor 140 and a comparison of the timing of continuously acquired ultrasound data 420 versus exemplary ECG triggered ultrasound data acquisitions 430, in accordance with various embodiments. Referring to FIG. 4, the display 400 comprises an ultrasound image display portion 402 with ECG signals 410 overlaid thereon. Typically, wearable ultrasound probes continuously acquire ultrasound data over an extended period of time. In various embodiments of the present application, the ECG signals 410 may be analyzed to determine a timing to trigger the ultrasound acquisitions 430, such as at end diastole and end systole. By selectively acquiring the ultrasound data when desired 430, as opposed to continuously 420, the wearable ultrasound probe 110 reduces power consumption and heating issues of the probe 110.

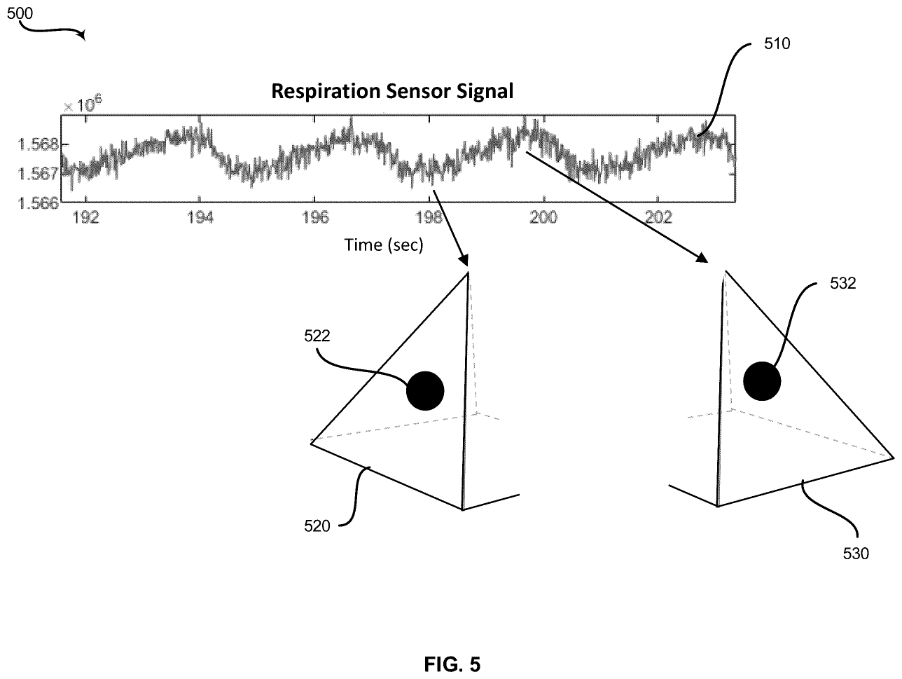

Referring again to FIGS. 1 and 2, a respiration sensor 140 may be operable to acquire respiration sensor signals. The respiration sensor signals may be analyzed by a processor 132, 152 to generate control signals for triggering a timing of ultrasound data acquisition and/or a targeted location of the ultrasound data acquisition based on the respiration sensor signals. The targeted location of the ultrasound data acquisition may be one of a plurality of targeted locations, where each of the plurality of targeted locations corresponds with a respiratory phase identified by the respiration sensor signals. FIG. 5 is an exemplary display 500 of a respiration sensor signal 510 acquired by a non-ultrasound sensor 140 and exemplary partial ultrasound volume acquisitions 520, 530 determined by a location of the target anatomical structure 522, 532 at different respiratory phases, in accordance with various embodiments. Referring to FIG. 5, the respiration sensor signal 510 may be acquired by a respiration sensor 140. A location of a target anatomical structure 522, 532 may change with the respiratory phases. For example, at a first time in a first respiration phase (e.g., at approximately 198 seconds), the location of the target anatomical structure 522 may be on a left side of a full volume of a region of interest. At a second time in a second respiration phase (e.g., at approximately 200 seconds), the location of the target anatomical structure 530 may have moved to a right side of a full volume of the region of interest. Typically, wearable ultrasound probes may continuously acquire a full ultrasound volume of the region of interest over an extended period of time, such that the target anatomical structure is acquired irrespective of the location at the particular respiration phase. In various embodiments of the present application, the respiration signals 510 may be analyzed to determine a timing to trigger the ultrasound acquisitions 520, 530. The respiration signals 510 may also be analyzed to determine a targeted location to perform the ultrasound acquisitions. For example, at a first time in a first respiration phase (e.g., at approximately 198 seconds), a first partial ultrasound volume 520 may be acquired based on the location of the target anatomical structure at the first time 522. At a second time in a second respiration phase (e.g., approximately 200 seconds), a second partial ultrasound volume 530 may be acquired based on the location of the target anatomical structure at the second time 532. By selectively acquiring partial ultrasound volumes 520, 530 at targeted locations and times, as opposed to continuously acquiring full ultrasound volumes, the wearable ultrasound probe 110 reduces power consumption and heating issues of the probe 110, and data storage and processing issues of the ultrasound system 100, 200.

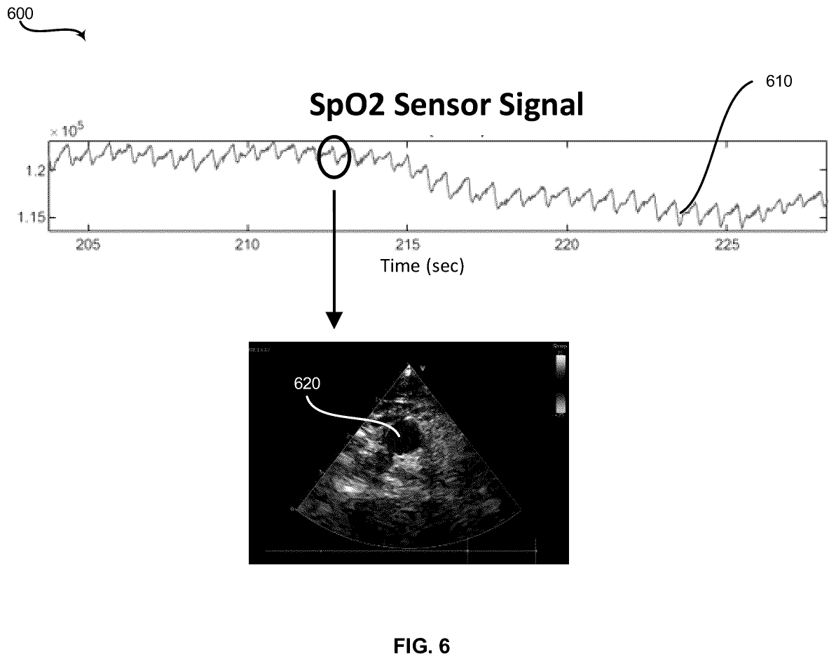

Referring again to FIGS. 1 and 2, a pulse oximeter sensor 140 may be operable to acquire local pulse oximeter sensor signals. The local pulse oximeter sensor signals may be analyzed by a processor 132, 152 to generate control signals for gating ultrasound data acquisitions. FIG. 6 is an exemplary display 600 of a local pulse oximeter sensor signal 610 acquired by a non-ultrasound sensor 140 and indicative of local blood flow 620 near a skin surface analyzed to determine gating of ultrasound acquisitions, in accordance with various embodiments. Referring to FIG. 6, the local pulse oximeter sensor signal 610 may be acquired by a pulse oximeter sensor 140. The local pulse oximeter sensor signal 610 may by analyzed by a processor 132, 152 to identify blood flow 620 near a skin surface. The control signals may be generated by the processor 132, 152 based on the local blood flow 620 near the skin surface to control the gating of the ultrasound data acquisitions.

Referring again to FIGS. 1 and 2, the probe processor 122 (if present) of the patient-worn probe transducer assembly may comprise suitable logic, circuitry, interfaces and/or code that may be operable to receive and apply the control signals for acquiring the ultrasound data. The control signals may be received from the control processor 132 of the patient-worn probe controller assembly 130, or from another processor (e.g., the signal processor 152 of the connected device 150 or a processor of the sensor(s) 140) via the control processor 132 of the patient-worn probe controller assembly 130. The probe processor 122 may be one or more Application-Specific Integrated Circuits (ASICs), one or more Field Programmable Gate Arrays (FPGA), one or more System on a Chip (SoC), and/or any suitable processing element. In various embodiments, the probe processor 122 may optionally comprise a transmit sub-aperture beamformer and/or a receive sub-aperture beamformer. For example, the control signals may be provided by the control processor 132 of the patient-worn probe controller assembly 130 to probe processor 122 of the patient-worn probe transducer assembly 120 which, through a transmit sub-aperture beamformer, drives the group of transmit transducer elements 124 to emit ultrasonic transmit signals into a region of interest (e.g., human, animal, underground cavity, physical structure and the like). In various embodiments, the transmit sub-aperture beamformer may not be included. The transmitted ultrasonic signals may be backscattered from structures in the object of interest, like blood cells or tissue, to produce echoes. The echoes are received by the receive transducer elements 126. The group of receive transducer elements 126 may be operable to convert the received echoes into electrical signals, which undergo sub-aperture beamforming by a receive sub-aperture beamformer. In various embodiments, the receive sub-aperture beamformer 116 may not be included. The probe processor 122 may be configured to apply the appropriate receive delays to the received signals as defined by the control signals received from the control processor 132 of the patient-worn probe controller assembly 130.

The probe processor 122 (if present) of the patient-worn probe transducer assembly 120, the control processor 132 of the patient-worn probe controller assembly 130, and/or a signal processor 152 of a connected device 150 may provide the functionality for processing the electrical signals to provide the resulting data output by the connected device 150. For example, the probe processor 122, control processor 132, and/or signal processor 152 may provide the functionality of a transmitter, transmit beamformer, receiver, receive beamformer, A/D converters, radio frequency (RF) processor, and/or display processor, among other things. In an exemplary embodiment, the probe processor 122, control processor 132, and/or signal processor 152 may be capable of executing any of the method(s) and/or set(s) of instructions discussed herein in accordance with the various embodiments, for example.

The probe processor 122 and/or control processor 132 may comprise suitable logic, circuitry, interfaces and/or code that may be operable to receive the signals from the receive sub-aperture beamformer and/or receive transducer elements 126. The analog signals may be processed by A/D converters of the probe processor 122 and/or control processor 132 to convert analog signals to corresponding digital signals. The probe processor 122 and/or control processor 132 may comprise suitable logic, circuitry, interfaces and/or code that may be operable to demodulate the digital signals output by the A/D converters. In accordance with an embodiment, the probe processor 122 and/or control processor 132 may comprise a complex demodulator that is operable to demodulate the digital signals to form in-phase and quadrature (IQ) data pairs that are representative of the corresponding echo signals. The RF or IQ signal data may then be temporarily stored in an RF/IQ buffer. The probe processor 122 and/or control processor 132 may comprise suitable logic, circuitry, interfaces and/or code that may be operable to perform digital beamforming processing to, for example, sum the delayed channel signals retrieved from the RF/IQ buffer and output a beam summed signal. The resulting processed information may be the beam summed signal that is communicated to the signal processor 152 of the connected device 150.

The patient-worn probe controller assembly 130 of the wearable ultrasound probe 110 may be communicatively coupled via a wired 190 and/or wireless 195 connection to a connected device 150. The connected device 150 may be an ultrasound console, a mobile device, and/or any suitable computing device. The connected device 150 may comprise a signal processor 152, a display system 154, an image buffer 156, an archive 158, and a user input device 160.

The user input device 160 may be utilized to input patient data, scan parameters, settings, select protocols and/or templates, and the like. In an exemplary embodiment, the user input device 160 may be operable to configure, manage and/or control operation of one or more components and/or modules in the ultrasound system 100, 200. The user input device 130 may include button(s), rotary encoder(s), a touchscreen, motion tracking, voice recognition, a mousing device, keyboard, camera and/or any other device capable of receiving a user directive. In certain embodiments, one or more of the user input devices 160 may be integrated into other components, such as the display system 154, for example. As an example, the user input device 160 may include a touchscreen display.

The signal processor 152 may comprise suitable logic, circuitry, interfaces and/or code that may be operable to process ultrasound scan data (i.e., summed IQ signal) for generating ultrasound images for presentation on a display system 154. The signal processor 152 is operable to perform one or more processing operations according to a plurality of selectable ultrasound modalities on the acquired ultrasound scan data. In an exemplary embodiment, the signal processor 152 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer during a scanning session and processed in less than real-time in a live or off-line operation. In various embodiments, the processed image data can be presented at the display system 154 and/or may be stored at the archive 158. The archive 158 may be a local archive, a Picture Archiving and Communication System (PACS), or any suitable device for storing images and related information.

The signal processor 152 may be one or more processing units, microprocessors, microcontrollers, GPU, and/or the like. The signal processor 152 may be an integrated component, or may be distributed across various locations, for example. The signal processor 152 may be capable of receiving input information from a user input device 160 and/or archive 158, generating an output displayable by a display system 154, and manipulating the output in response to input information from a user input device 160, among other things.

The ultrasound system 100, 200 may be operable to continuously and/or selectively (i.e., intelligently) acquire ultrasound scan data at a frame rate that is suitable for the imaging situation in question. Typical frame rates range from 20-120 but may be lower or higher. The acquired ultrasound scan data may be displayed on the display system 154 at a display-rate that can be the same as the frame rate, or slower or faster. An image buffer 156 is included for storing processed frames of acquired ultrasound scan data that are not scheduled to be displayed immediately. Preferably, the image buffer 156 is of sufficient capacity to store at least several minutes' worth of frames of ultrasound scan data but it can also store less. The frames of ultrasound scan data are stored in a manner to facilitate retrieval thereof according to its order or time of acquisition. The image buffer 156 may be embodied as any known data storage medium.

Still referring to FIGS. 1 and 2, the display system 154 may be any device capable of communicating visual information to a user. For example, a display system 154 may include a liquid crystal display, a light emitting diode display, and/or any suitable display or displays. The display system 154 can be operable to present ultrasound images, metrics, and/or any suitable information.

The archive 158 may be one or more computer-readable memories integrated with the ultrasound system 100, 200 and/or communicatively coupled (e.g., over a network) to the ultrasound system 100, 200, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 158 may include databases, libraries, sets of information, or other storage accessed by and/or incorporated with the signal processor 152, for example. The archive 158 may be able to store data temporarily or permanently, for example. The archive 158 may be capable of storing medical image data, data generated by the signal processor 152, and/or instructions readable by the signal processor 152, among other things.

In certain embodiments, the connected device 150 may include output device(s) in addition to or as an alternative to the display system 154, such as an audio system, haptic feedback system, and/or any suitable output system. In an exemplary embodiment, the connected device 150 may be communicatively coupled to external systems, such as a server (e.g., cloud server), network (e.g., hospital network), and/or any suitable external system.

Components of the ultrasound system 100, 200 may be implemented in software, hardware, firmware, and/or the like. The various components of the ultrasound system 100, 200 may be communicatively linked. Components of the ultrasound system 100, 200 may be implemented separately and/or integrated in various forms. For example, the display system 154 and the user input device 160 may be integrated as a touchscreen display.

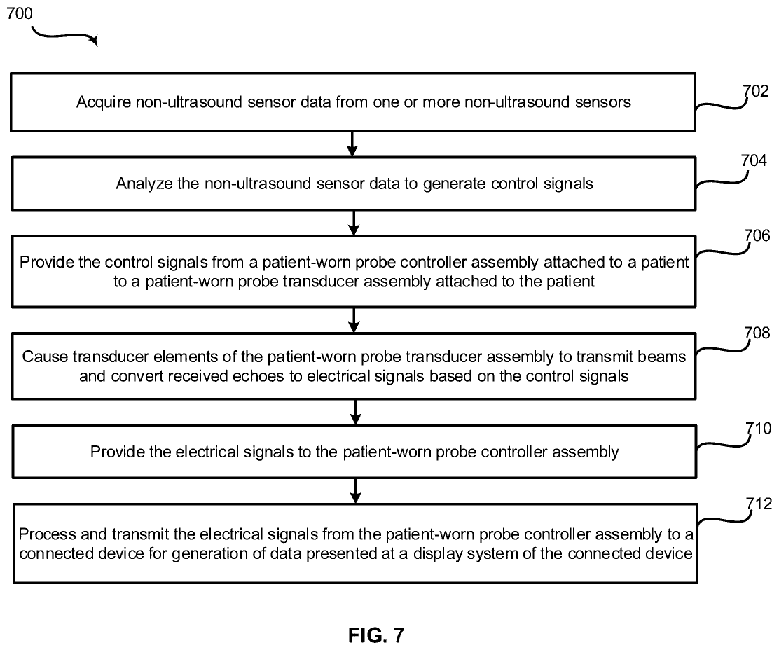

FIG. 7 is a flow chart 700 illustrating exemplary steps 702-712 that may be utilized for performing intelligent ultrasound data acquisition with a wearable ultrasound probe 110 having electronics 122, 124, 126, 132, 140 distributed between a patient-worn probe transducer assembly 120 and a patient-worn probe controller assembly 130, in accordance with various embodiments. Referring to FIG. 7, there is shown a flow chart 700 comprising exemplary steps 702 through 712. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 702, one or more non-ultrasound sensors 140 of a wearable ultrasound probe 110 of an ultrasound system 100, 200 may acquire non-ultrasound sensor data 410, 510, 610. For example, the one or more non-ultrasound sensors 140 may comprise electrocardiogram (ECG) sensor(s) 140 operable to acquire ECG signals, respiration sensor(s) 140 operable to acquire respiration sensor signals, pulse oximeter sensor(s) 140 operable to acquire local pulse oximeter sensor signals, motion sensor(s) 140 operable to acquire patient posture sensor signals, temperature sensor(s) 140 operable to acquire temperature sensor signals, blood pressure sensor(s) 140 operable to acquire blood pressure sensor signals, and/or any suitable non-ultrasound sensor.

At step 704, a processor 132, 152 analyzes the non-ultrasound sensor data 410, 510, 610 to generate control signals. For example, the non-ultrasound sensor data 410, 510, 610 acquired at step 702 may be analyzed by one or more processors 132, 152 to generate control signals for acquiring ultrasound data. As an example, ECG signals 410 may be analyzed to generate control signals for triggering a timing of ultrasound data acquisition. As another example, respiratory sensor signals 510 may be analyzed to generate control signals for triggering a timing of ultrasound data acquisition and/or a targeted location of the ultrasound data acquisition based on the respiration sensor signals. The targeted location of the ultrasound data acquisition may be one of a plurality of targeted locations, where each of the plurality of targeted locations corresponds with a respiratory phase identified by the respiration sensor signals. As another example, local pulse oximeter sensor signals 610 may be analyzed to generate control signals for gating ultrasound data acquisitions. In various embodiments, patient posture sensor signals may be analyzed to flag unreliable ultrasound acquisitions and/or to provide the motion information to a connected device 150. In certain embodiments, temperature sensor signals may be analyzed to provide temperature information to the connected device 150. In an exemplary embodiment, blood pressure sensor signals may be analyzed to provide blood pressure information to the connected device 150.

At step 706, at least one control processor 132 of the patient-worn probe controller assembly 130 attached to a patient provides the control signals to a patient-worn probe transducer assembly 120 attached to the patient. For example, at least one control processor 132 of the patient-worn probe controller assembly 130 may generate and/or receive the control signals generated at step 704. The at least one control processor 132 communicates the control signals to the patient-worn probe transducer assembly 120 via a probe cable 180.

At step 708, transducer elements 124, 126 of the patient-worn probe transducer assembly 120 transmit beams and convert received echoes to electrical signals based on the control signals received from the at least one control processor 132 of the patient-worn probe controller assembly 130. For example, the patient-worn probe transducer assembly 120 may be configured to apply the control signals to drive the transmit transducer elements 124 to emit ultrasonic transmit beams into a region of interest. The transmitted ultrasonic beams may be backscattered from structures in the region of interest to produce echoes. The echoes are received by the receive transducer elements 126 of the patient-worn probe transducer assembly 120, which may constitute the same transducer elements as the transmit transducer elements 124. The receive transducer elements 126 in the patient-worn probe transducer assembly 120, in some cases under the control of the probe processor 122, may be operable to convert the received echoes into electrical signals. The probe processor 122 may comprise one or more Application-Specific Integrated Circuits (ASICs) or any suitable processing element. In various embodiments, the at least one probe processor 122 may comprise a transmit sub-aperture beamformer and a receive sub-aperture beamformer. The at least one probe processor 122 may be configured to drive the transmit transducer elements 124 to emit the ultrasonic transmit beams via the transmit sub-aperture beamformer. The at least one probe processor 122 may be configured to apply the electrical signals generated by the receive transducer elements 126 to the receive sub-aperture beamformer to undergo sub-aperture beamforming.

At step 710, the patient-worn probe transducer assembly 120 may provide the electrical signals to the patient-worn probe controller assembly 130. For example, the probe controller 122 of the patient-worn probe transducer assembly 120 may communicate the electrical signals generated at step 708 to the at least one control processor 132 of the patient-worn probe controller assembly 130 via the probe cable 180.

At step 712, the at least one control processor 132 of the patient-worn probe controller assembly 130 processes and transmits the electrical signals to a connected device 150 for generation of data to present at a display system 154 of the connected device 150. For example, the electrical signals may be processed by the at least one control processor 132 to perform A/D conversion, RF processing, and/or receive beamforming. Additionally or alternatively, some or all of the A/D conversion, RF processing, and/or receive beamforming may be performed by the probe processor 122 of the patient-worn probe transducer assembly 120 and/or a signal processor 152 of the connected device 150. The processor(s) 122, 132, 152 may convert analog signals to corresponding digital signals. The digital signals may be demodulated to form IQ data pairs that are representative of the corresponding echo signals. The RF or IQ signal data may then be communicated to an RF/IQ buffer. The RF/IQ buffer may be operable to provide temporary storage of the RF or IQ signal data. The processor(s) 122, 132, 152 may perform digital beamforming processing to, for example, sum the delayed channel signals received via the RF/IQ buffer and output a beam summed signal. The resulting processed information may be the beam summed signal that is communicated to a signal processor 152 of the connected device 150. The signal processor 152 may process the ultrasound scan data (i.e., summed IQ signal) to generate ultrasound images, which may be presented at a display system 154 and/or may be stored at an archive 158. The archive 158 may be a local archive, a Picture Archiving and Communication System (PACS), or any suitable device for storing images and related information. The connected device 150 may be communicatively coupled to the patient-worn probe controller assembly 130 of the wearable ultrasound probe 110 by a wired 190 or wireless 195 connection. The connected device 150 may be an ultrasound console, a mobile device, and/or any suitable computing device.

In certain embodiments, the output of the connected device 150 may be an ultrasound image, a metric, and/or any suitable output. In embodiments where an ultrasound image is not presented and/or stored, the metric or other output may be provided by a display system 154 or some other output system, such as an audio system, haptic feedback system, or any suitable output system. In an exemplary embodiment, the connected device may be communicatively coupled to external systems, such as a server (e.g., cloud server), network (e.g., hospital network), and/or any suitable external system.

In an exemplary embodiment, a simplified ultrasound acquisition from the patient-worn probe transducer assembly 120 may be used to control a full ultrasound acquisition from the patient-worn probe transducer assembly 120. For example, a single beam or some sparsely sampled beams from the patient-worn probe transducer assembly 120 could be used to monitor the subject, and when appropriate, trigger a full acquisition. In this case, instead of or in addition to using separate, non-ultrasound sensors, the patient-worn probe transducer assembly 120 may be used to intermittently transmit and receive ultrasound signals in a low power consumption mode in between full acquisitions.

Aspects of the present disclosure provide a method 700 and system 100, 200 for performing intelligent ultrasound data acquisition with a wearable ultrasound probe 110 having electronics 122, 124, 126, 132, 140 distributed between a patient-worn probe transducer assembly 120 and a patient-worn probe controller assembly 130. In accordance with various embodiments, the wearable ultrasound probe 110 may comprise a patient-worn probe transducer assembly 120 and a patient-worn probe controller assembly 130. The patient-worn probe transducer assembly 120 may comprise transducer elements 124, 126 disposed in a first housing. The transducer elements 124, 126 may be configured to transmit ultrasound beams and convert received echoes to electrical signals in response to control signals. The patient-worn probe controller assembly 130 may comprise at least one control processor 132 disposed in a second housing. The at least one control processor 132 may be communicatively coupled 190, 195 to a connected device 150. The at least one control processor 132 may be communicatively coupled 180 to the patient-worn probe transducer assembly 120 via a wired connection 180. The at least one control processor 132 may be configured to provide the control signals to the patient-worn probe transducer assembly 120 for controlling ultrasound data acquisition.

In an exemplary embodiment, the wearable ultrasound probe 110 is a four-dimensional (4D) probe 110. The patient-worn probe transducer assembly 120 may further comprise at least one probe processor 122. The at least one probe processor 122 may be configured to cause the transducer elements 124, 126 to transmit the ultrasound beams and convert the received echoes to electrical signals in response to the control signals. In a representative embodiment, the at least one control processor 132 may be communicatively coupled 190, 195 to the connected device 150 via a wireless connection 195. One or both of the patient-worn probe controller assembly 130 and the patient-worn probe transducer assembly 120 may comprise a battery 170 configured to provide power for the wearable ultrasound probe 110. In various embodiments, the second housing is a strain relief. In certain embodiments, the at least one probe processor 122 comprises an Application-Specific Integrated Circuit (ASIC). In an exemplary embodiment, each of the patient-worn probe transducer assembly 120 and the patient-worn probe controller assembly 130 is configured to be attached to a patient via a patient attachment mechanism 105 comprising fixation adhesive 105, a strap, and/or a harness. In a representative embodiment, the wearable ultrasound probe 110 comprises one or more non-ultrasound sensors 140. Each of the one or more non-ultrasound sensors 140 may be communicatively coupled to the at least one control processor 132 and may be provided in the first housing of the patient-worn probe transducer assembly 120, in the second housing of the patient-worn probe controller assembly 130 and/or in a third housing of a patient-worn sensor electronics assembly.

In various embodiments, the one or more non-ultrasound sensors 140 comprises an electrocardiogram (ECG) sensor 140 operable to acquire ECG signals 410. The at least one control processor 132 may be configured to provide the control signals communicated to the patient-worn probe transducer assembly 120 for triggering a timing 430 of ultrasound data acquisition based on the ECG signals 410. In certain embodiments, the one or more non-ultrasound sensors 140 comprises a respiration sensor 140 operable to acquire respiration sensor signals 510. The at least one control processor 132 may be configured to provide the control signals communicated to the patient-worn probe transducer assembly 120 for triggering a timing of ultrasound data acquisition and/or a targeted location 522, 532 of the ultrasound data acquisition 520, 530 based on the respiration sensor signals 510. The targeted location 522, 532 of the ultrasound data acquisition 520, 530 may be one of a plurality of targeted locations 522, 532. Each of the plurality of targeted locations 522, 532 may correspond with a respiratory phase identified by the respiration sensor signals 510. In an exemplary embodiment, the one or more non-ultrasound sensors 140 comprises a pulse oximeter sensor 140 operable to acquire local pulse oximeter sensor signals 610. The at least one control processor 132 may be configured to provide the control signals communicated to the patient-worn probe transducer assembly 120 for gating ultrasound data acquisitions based on the local pulse oximeter sensor signals 610.

In a representative embodiment, the one or more non-ultrasound sensors 140 comprises a motion sensor 140 operable to acquire patient posture sensor signals for flagging unreliable ultrasound data and/or determining motion information. The one or more non-ultrasound sensors 140 may comprise a temperature sensor 140 operable to acquire temperature sensor signals for determining temperature information. The one or more non-ultrasound sensors 140 may comprise a blood pressure sensor 140 operable to acquire blood pressure sensor signals for determining blood pressure information.

Various embodiments provide an ultrasound system 100, 200 operable to perform intelligent ultrasound data acquisition with a wearable ultrasound probe 110 having electronics 122, 124, 126, 132, 140 distributed between a patient-worn probe transducer assembly 120 and a patient-worn probe controller assembly 130. The ultrasound system 100, 200 may comprise a connected device 150 and a four-dimensional (4D) wearable ultrasound probe 110. The four-dimensional (4D) wearable ultrasound probe 110 may comprise a patient-worn probe transducer assembly 120 and a patient-worn probe controller assembly 130. The patient-worn probe transducer assembly may comprise at least one probe processor 122 and transducer elements 124, 126 disposed in a first housing. The at least one probe processor 122 may be configured to cause the transducer elements 124, 126 to transmit ultrasound beams and convert received echoes to electrical signals in response to control signals. The patient-worn probe controller assembly 130 may comprise at least one control processor 132 disposed in a second housing. The at least one control processor 132 may be communicatively coupled 190, 195 to the connected device 150. The at least one control processor 132 may be communicatively coupled 180 to the at least one probe processor 122 of the patient-worn probe transducer assembly 120 via a wired connection 180. The at least one control processor 132 may be configured to provide the control signals to the at least one probe processor 122 for controlling ultrasound data acquisition.

In a representative embodiment, the at least one control processor 132 is communicatively coupled to the connected device 150 via a wireless connection 195. One or more of the patient-worn probe controller assembly 130 and the patient-worn probe transducer assembly 120 comprises a battery 170 configured to provide power to the 4D wearable ultrasound probe 110. In various embodiments, the second housing is a strain relief. The at least one probe processor 122 may comprise an Application-Specific Integrated Circuit (ASIC). In certain embodiments, each of the patient-worn probe transducer assembly 120 and the patient-worn probe controller assembly 130 is configured to be attached to a patient via a patient attachment mechanism 105 comprising fixation adhesive 105, a strap, and/or a harness. In an exemplary embodiment, the ultrasound system 100, 200 comprises one or more non-ultrasound sensors 140. Each of the one or more non-ultrasound sensors 140 may be communicatively coupled to the at least one control processor 132 and is provided in the first housing of the patient-worn probe transducer assembly 120, in the second housing of the patient-worn probe controller assembly 130, and/or in a third housing of a patient-worn sensor electronics assembly. In a representative embodiment, the one or more non-ultrasound sensors 140 comprises an electrocardiogram (ECG) sensor 140 operable to acquire ECG signals 410. The at least one control processor 132 may be configured to provide the control signals communicated to the at least one probe processor 122 for triggering a timing 430 of ultrasound data acquisition based on the ECG signals 410. In various embodiments, the one or more non-ultrasound sensors 140 comprises a respiration sensor 140 operable to acquire respiration sensor signals 510. The at least one control processor 132 may be configured to provide the control signals communicated to the at least one probe processor 122 for triggering a timing of ultrasound data acquisition and/or a targeted location 522, 532 of the ultrasound data acquisition 520, 530 based on the respiration sensor signals 510. The targeted location 522, 532 of the ultrasound data acquisition 520, 530 may be one of a plurality of targeted locations 522, 532. Each of the plurality of targeted locations 522, 532 may correspond with a respiratory phase identified by the respiration sensor signals 510. In an exemplary embodiment, the one or more non-ultrasound sensors 140 comprises a pulse oximeter sensor 140 operable to acquire local pulse oximeter sensor signals 610. The at least one control processor 132 may be configured to provide the control signals communicated to the at least one probe processor 122 for gating ultrasound data acquisitions based on the local pulse oximeter sensor signals 610.

Certain embodiments provide a method 700 for performing intelligent ultrasound data acquisition with a wearable ultrasound probe 110 having electronics 122, 124, 126, 132, 140 distributed between a patient-worn probe transducer assembly 120 and a patient-worn probe controller assembly 130. The method 700 may comprise receiving 706 control signals at a patient-worn probe transducer assembly 120 comprising transducer elements 124, 126 disposed in a first housing. The control signals may be provided via a wired connection 180 to the patient-worn probe transducer assembly 120 from a patient-worn probe controller assembly 130 comprising at least one control processor 132 disposed in a second housing. Each of the patient-worn probe transducer assembly 120 and the patient-worn probe controller assembly 130 may be attached to a patient via a patient attachment mechanism 105 comprising fixation adhesive, a strap, and/or a harness. The method 700 may comprise causing 708 the transducer elements 124, 126 to transmit ultrasound beams and convert received echoes to electrical signals in response to control signals. The electrical signals may be provided 710 by the patient-worn probe transducer assembly 120 to the at least one control processor 132 of the patient-worn probe controller assembly 130. The method 700 may comprise processing and transmitting 712, by the at least one control processor 132 of the patient-worn probe controller assembly 130, the electrical signals to a connected device 150 communicatively coupled 190, 195 to the patient-worn probe controller assembly 130 for generation of data presented at a display system 154 of the connected device 150. The control signals may be provided by the at least one control processor 132 of the patient-worn probe controller assembly 130 based on non-ultrasound sensor data 410, 510, 610. The control signals may be configured to trigger a timing 430 of ultrasound data acquisition based on electrocardiogram (ECG) sensor signals 410. Additionally and/or alternatively, the control signals may be configured to trigger a timing of the ultrasound data acquisition and/or a targeted location 522, 532 of the ultrasound data acquisition 520, 530 based on respiration sensor signals 510. The targeted location 522, 532 of the ultrasound data acquisition 520, 530 may be one of a plurality of targeted locations 522, 532. Each of the plurality of targeted locations 522, 532 may correspond with a respiratory phase identified by the respiration sensor signals 510. Additionally and/or alternatively, the control signals may be configured to gate the ultrasound data acquisition based on local pulse oximeter sensor signals 610.

As utilized herein the term “circuitry” refers to physical electronic components (i.e., hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” and/or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for performing intelligent ultrasound data acquisition with a wearable ultrasound probe having electronics distributed between a patient-worn probe transducer assembly and a patient-worn probe controller assembly.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A wearable ultrasound probe comprising:
a patient-worn probe transducer assembly comprising transducer elements disposed in a first housing, the transducer elements configured to transmit ultrasound beams and convert received echoes to electrical signals in response to control signals; and
a patient-worn probe controller assembly comprising at least one control processor disposed in a second housing, the at least one control processor communicatively coupled to a connected device, the at least one control processor communicatively coupled to the patient-worn probe transducer assembly via a wired connection, the at least one control processor configured to provide the control signals to the patient-worn probe transducer assembly for controlling ultrasound data acquisition.

2. The wearable ultrasound probe of claim 1, wherein:
the wearable ultrasound probe is a four-dimensional (4D) probe, and
the patient-worn probe transducer assembly further comprises at least one probe processor, the at least one probe processor configured to cause the transducer elements to transmit the ultrasound beams and convert the received echoes to electrical signals in response to the control signals.

3. The wearable ultrasound probe of claim 1, wherein:
the at least one control processor is communicatively coupled to the connected device via a wireless connection; and
one or both of the patient-worn probe controller assembly and the patient-worn probe transducer assembly comprises a battery configured to provide power for the wearable ultrasound probe.

4. The wearable ultrasound probe of claim 1, wherein the second housing is a strain relief.

5. The wearable ultrasound probe of claim 2, wherein the at least one probe processor comprises an Application-Specific Integrated Circuit (ASIC).

6. The wearable ultrasound probe of claim 1, wherein each of the patient-worn probe transducer assembly and the patient-worn probe controller assembly is configured to be attached to a patient via a patient attachment mechanism comprising:
fixation adhesive,
a strap, and/or
a harness.

7. The wearable ultrasound probe of claim 1, comprising one or more non-ultrasound sensors, wherein each of the one or more non-ultrasound sensors is communicatively coupled to the at least one control processor and is provided:
in the first housing of the patient-worn probe transducer assembly;
in the second housing of the patient-worn probe controller assembly; and/or
in a third housing of a patient-worn sensor electronics assembly.

8. The wearable ultrasound probe of claim 7, wherein:
the one or more non-ultrasound sensors comprises an electrocardiogram (ECG) sensor operable to acquire ECG signals; and
the at least one control processor is configured to provide the control signals communicated to the patient-worn probe transducer assembly for triggering a timing of ultrasound data acquisition based on the ECG signals.

9. The wearable ultrasound probe of claim 7, wherein:
the one or more non-ultrasound sensors comprises a respiration sensor operable to acquire respiration sensor signals; and
the at least one control processor is configured to provide the control signals communicated to the patient-worn probe transducer assembly for triggering a timing of ultrasound data acquisition and/or a targeted location of the ultrasound data acquisition based on the respiration sensor signals, wherein the targeted location of the ultrasound data acquisition is one of a plurality of targeted locations, each of the plurality of targeted locations corresponding with a respiratory phase identified by the respiration sensor signals.

10. The wearable ultrasound probe of claim 7, wherein:
the one or more non-ultrasound sensors comprises a pulse oximeter sensor operable to acquire local pulse oximeter sensor signals; and
the at least one control processor is configured to provide the control signals communicated to the patient-worn probe transducer assembly for gating ultrasound data acquisitions based on the local pulse oximeter sensor signals.

11. The wearable ultrasound probe of claim 7, wherein the one or more non-ultrasound sensors comprises:
a motion sensor operable to acquire patient posture sensor signals for flagging unreliable ultrasound data and/or determining motion information;
a temperature sensor operable to acquire temperature sensor signals for determining temperature information; and/or
a blood pressure sensor operable to acquire blood pressure sensor signals for determining blood pressure information.

12. An ultrasound system comprising:
a connected device; and
a four-dimensional (4D) wearable ultrasound probe comprising:
a patient-worn probe transducer assembly comprising at least one probe processor and transducer elements disposed in a first housing, the at least one probe processor configured to cause the transducer elements to transmit ultrasound beams and convert received echoes to electrical signals in response to control signals; and
a patient-worn probe controller assembly comprising at least one control processor disposed in a second housing, the at least one control processor communicatively coupled to the connected device, the at least one control processor communicatively coupled to the at least one probe processor of the patient-worn probe transducer assembly via a wired connection, the at least one control processor configured to provide the control signals to the at least one probe processor for controlling ultrasound data acquisition.

13. The ultrasound system of claim 12, wherein:
the at least one control processor is communicatively coupled to the connected device via a wireless connection; and
one or more of the patient-worn probe controller assembly and the patient-worn probe transducer assembly comprises a battery configured to provide power to the 4D wearable ultrasound probe.

14. The ultrasound system of claim 12, wherein:
the second housing is a strain relief; and/or
the at least one probe processor comprises an Application-Specific Integrated Circuit (ASIC).

15. The ultrasound system of claim 12, wherein each of the patient-worn probe transducer assembly and the patient-worn probe controller assembly is configured to be attached to a patient via a patient attachment mechanism comprising:
fixation adhesive,
a strap, and/or
a harness.

16. The ultrasound system of claim 12, comprising one or more non-ultrasound sensors, wherein each of the one or more non-ultrasound sensors communicatively coupled to the at least one control processor and is provided:
in the first housing of the patient-worn probe transducer assembly;
in the second housing of the patient-worn probe controller assembly; and/or
in a third housing of a patient-worn sensor electronics assembly.

17. The ultrasound system of claim 16, wherein:
the one or more non-ultrasound sensors comprises an electrocardiogram (ECG) sensor operable to acquire ECG signals; and
the at least one control processor is configured to provide the control signals communicated to the at least one probe processor for triggering a timing of ultrasound data acquisition based on the ECG signals.

18. The ultrasound system of claim 16, wherein:
the one or more non-ultrasound sensors comprises a respiration sensor operable to acquire respiration sensor signals; and
the at least one control processor is configured to provide the control signals communicated to the at least one probe processor for triggering a timing of ultrasound data acquisition and/or a targeted location of the ultrasound data acquisition based on the respiration sensor signals, wherein the targeted location of the ultrasound data acquisition is one of a plurality of targeted locations, each of the plurality of targeted locations corresponding with a respiratory phase identified by the respiration sensor signals.

19. The ultrasound system of claim 16, wherein:
the one or more non-ultrasound sensors comprises a pulse oximeter sensor operable to acquire local pulse oximeter sensor signals; and
the at least one control processor is configured to provide the control signals communicated to the at least one probe processor for gating ultrasound data acquisitions based on the local pulse oximeter sensor signals.

20. A method comprising:
receiving control signals at a patient-worn probe transducer assembly comprising transducer elements disposed in a first housing, wherein the control signals are provided via a wired connection to the patient-worn probe transducer assembly from a patient-worn probe controller assembly comprising at least one control processor disposed in a second housing, wherein each of the patient-worn probe transducer assembly and the patient-worn probe controller assembly is attached to a patient via a patient attachment mechanism comprising fixation adhesive, a strap, and/or a harness;
causing the transducer elements to transmit ultrasound beams and convert received echoes to electrical signals in response to control signals, wherein the electrical signals are provided by the patient-worn probe transducer assembly to the at least one control processor of the patient-worn probe controller assembly;
processing and transmitting, by the at least one control processor of the patient-worn probe controller assembly, the electrical signals to a connected device communicatively coupled to the patient-worn probe controller assembly for generation of data presented at a display system of the connected device,
wherein:
the control signals are provided by the at least one control processor of the patient-worn probe controller assembly based on non-ultrasound sensor data; and
the control signals are configured to:
trigger a timing of ultrasound data acquisition based on electrocardiogram (ECG) sensor signals;
trigger a timing of the ultrasound data acquisition and/or a targeted location of the ultrasound data acquisition based on respiration sensor signals, wherein the targeted location of the ultrasound data acquisition is one of a plurality of targeted locations, each of the plurality of targeted locations corresponding with a respiratory phase identified by the respiration sensor signals; and/or
gate the ultrasound data acquisition based on local pulse oximeter sensor signals.
